(19) United States
(12) Patent Application Publication
(10) Pub. No.: US 2024/0374736 A1
(43) Pub. Date: Nov. 14, 2024

(54) INHALED LIQUID FOR THE TREATMENT OF RESPIRATORY INFECTIONS IN HUMANS CAUSED BY PATHOGENS RESISTANT TO ANTIBIOTICS

(71) Applicant: Dusan Ninkov, San Diego, CA (US)

(72) Inventor: Dusan Ninkov, San Diego, CA (US)

(21) Appl. No.: 18/196,962

(22) Filed: May 12, 2023

(51) Int. Cl. A61K 47/55; A61K 9/00; A61P 31/04

(52) U.S. Cl. CPC A61K 47/55; A61K 9/0078; A61P 31/04

(57) ABSTRACT

The invention relates to a novel compound manufactured with the procedure of synthesis of bouncing Eugenol and Lidocaine into one stable compound. Use of the compound as an active compound/potent antimicrobial for the treatment of respiratory infections in humans caused by pathogens resistant to antibiotics, such as Pasteurella spp., Cryptococcus spp., Campylobacter spp., Microsporidia spp., Histoplasma capsulatum, Micrococcus spp., Candida spp., and other bacteria.

### FIELD OF THE INVENTION

The compound is a novel medicine for treating respiratory infections in humans.

### BACKGROUND OF THE INVENTION

This invention addresses the problems of respiratory infections in humans with the different etiology of pathogens. The compound is a broad-spectrum antimicrobial compound as a liquid for inhalation to treat respiratory infections. The antimicrobial effect of Eugenol is well known. This invention is completely novel because Eugenol and Lidocaine, by the special process of synthesis, have been bounced into one stable compound. The effect and uniqueness of this compound are the subject of this invention. The active principle of the compound is a stable quaternary ammonium complex of Eugenol and Lidocaine in the form of an inhaled liquid. The compound is more effective than standard 3rd generation cephalosporin antibiotics in treating mentioned infections. The compound is created to fight antibiotic resistance of pathogenic bacteria, such as Pasteurella spp., Cryptococcus spp., Campylobacter spp., Microsporidia spp., Histoplasma capsulatum, Micrococcus spp., Candida spp., and other bacteria causing respiratory infections in humans. Based on the specific mechanism of action, the active compound of this invention is different from any other antibiotic currently available on the market. In this invention, an extra potent effect of the synergy of Eugenol and Lidocaine is achieved. The antimicrobial efficacy of this novel compound will be significantly more effective than Eugenol and Lidocaine by themselves or by their simple mixture.

Before the widespread use of Penicillin, infectious diseases were the leading cause of death among all age groups across the globe. Antibiotics have extended lives and improved the quality of life of people since the early 20^{th} century. Many have thought that humanity was entering the post-pathogen era, where infectious disease was a historical footnote. Today, infectious diseases are still the first leading cause of death and the leading cause of disability.

### DESCRIPTION OF THE INVENTION

In the following text, the novelty and uniqueness of the compound will be elaborated.

### METHOD OF PRODUCTION

The novel compound is synthesized by reacting Eugenol (Sigma Aldrich) and an equimolar mass of free base Lidocaine (Sigma Aldrich) in a Dichloromethane solvent. After the reaction of those two chemicals at room temperature, for 30 min. a rotary vacuum evaporator is used to evaporate Dichloromethane from the mixture, leaving us with the pure novel compound. The novel compound is a quaternary ammonium complex salt of the naturally existing phenolic compound—Eugenol, with a synthetic local anesthetic compound—Lidocaine as a free base form. The ratio of Lidocaine to Eugenol in this reaction is in the range between 1:1 and 2:1

### Schematic Diagram of Synthesis Procedure

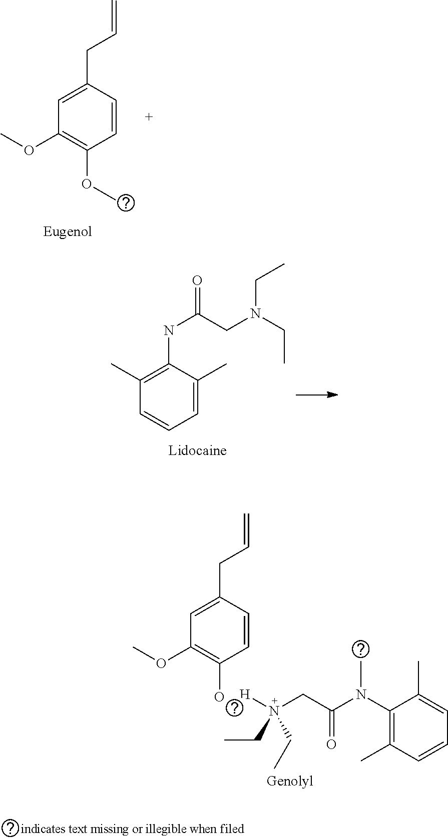

### Structural Formula of the Compound

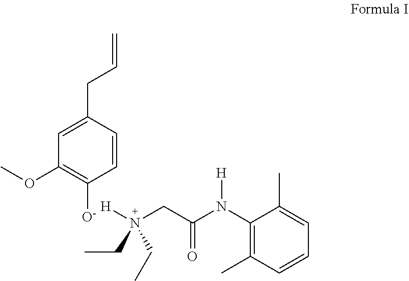

Chemical formula: C₂₄H₄₃N₃O₃

Molecular mass: 398.54 g mol⁻¹

Compound chemical name:
N-[N′-(carboxymethylenyl-2,6-dimethyl-aniline)-N,N-diethylammonium]-[2′-methoxy-4′-(2′-propenyl)]-phenolate.

### Characterization

Gas-mass and IIPLC chromatography can be used to characterize the new complex. In polar solvents such as water-methanol systems, two separate peaks of the complex constituents should be obtained, Lidocaine as a cation and Eugenol as an anion. Polar solvents tend to separate anions from cations in solution, which is why two separate peaks in such a system can be observed. Each peak is characterized by a mass spectrometer, and the information obtained proves their origin from Eugenol and Lidocaine. The new complex can also be characterized by 1H-NMR and IR spectra.

### BRIEF DESCRIPTION OF THE DRAWINGS

For a more complete understanding of the invention, reference is made to the following description and accompanying drawings, in which:

FIG. 1 Eugenol 1H NMR spectrum analysis, obtained from a reputable chemical database (ChemNMR).;

FIG. 2 Lidocaine III NMR spectrum analysis, obtained from a reputable chemical database (ChemNMRI).;

FIG. 3 The compounds 1H NMR spectrum analysis, obtained after the synthesis of the novel compound.:

FIG. 4 The compounds IR spectrum analysis, obtained after the synthesis of the novel compound.; and

FIG. 5 Lidocaine IR spectrum analysis, obtained from a reputable chemical database (NIST Chemistry WebBook).

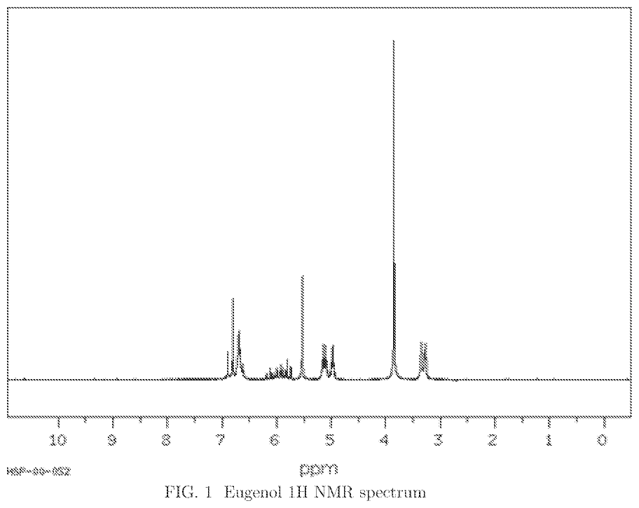

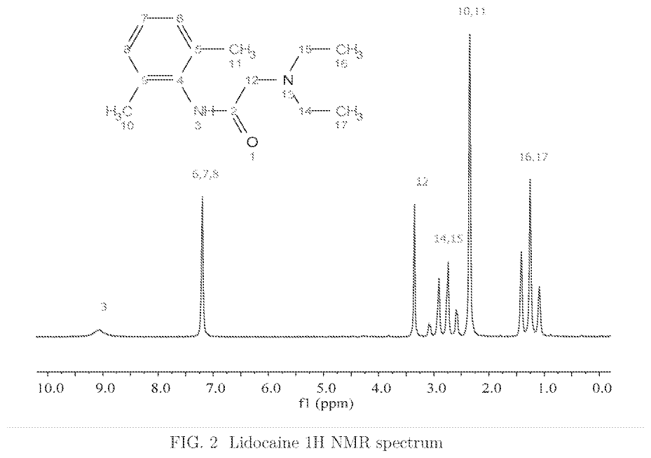

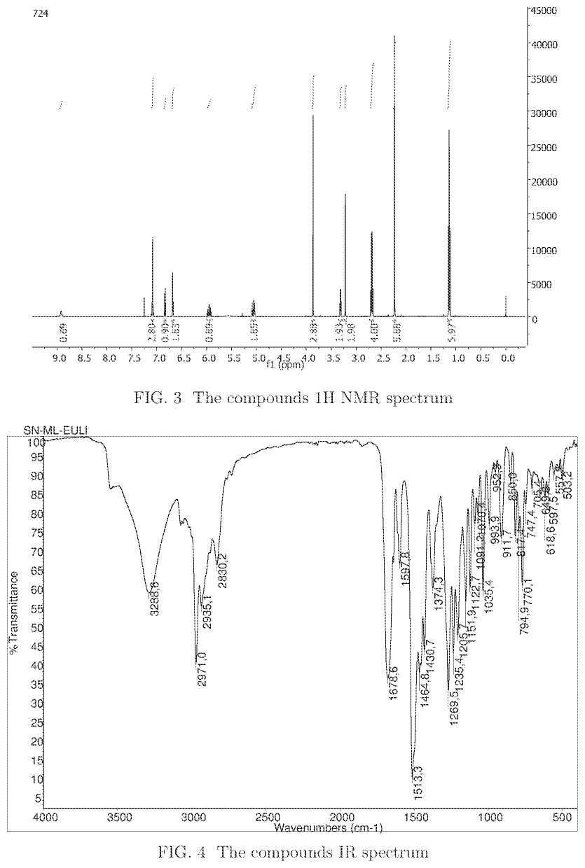

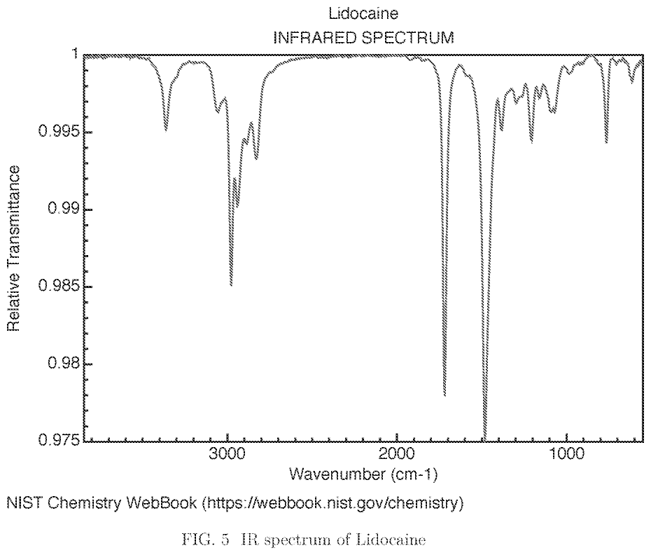

### Quality Control

In 1H-NMR spectra, the difference between free Eugenol characteristic positions of H-atoms and the same position of corresponding H-atoms of free Eugenol from a database was compared. The different positions of those H-atoms would conclude that the obtained compound is new, a combination of Eugenol and Lidocaine. Additionally, the IR spectra of the novel compound were also measured, proving that the novel compound is, in fact, a novel by not having the spectra data in any reputable database.

Eugenol is colorless to pale yellow oily liquid at room temperature. It can be found in many essential oils from natural products such as clove oil, nutmeg, cinnamon, basil bay leaf oil and possess antiseptic and anesthetic properties. It is also a well-known antibacterial, antifungal, antimicrobial, and anticoagulant agent. Uses of Eugenol have a wide spectrum, and its potential for medical uses is tremendous.

### Manufacturing of the Compound

This text briefly describes how the compound will be manufactured.

Formula:

| | | |
|---|---|---|
| Water: | 90 | mL |
| Emulsifier: | 8 | mL |
| Novel active compound: | 2 | mL |
| TOTAL: | 100 | mL |

The compound was obtained by mixing the above-stated ingredients in a vacuum mixer (Silverson L5M-A Sealed Unit Lab Mixer) at 6.000 rpm for 20 min.

### Treatment of Respiratory Infections in Humans

Case no. 1: Y. D., Male, Age 55, USA, Albuquerque Symptoms: Acute Bronchitis
Case no. 2: I. M., Male, Age 24, USA, Madison Symptoms: Acute Bronchitis
Case no. 3: F. P., Male, Age 59, USA, Greensboro Symptoms: Pneumonia
Case no. 4: A. Y., Male. Age 50, USA, Bakersfield Symptoms: Bronchopneumonia
Case no. 5: J. D., Male, Age 38, USA. Fort Worth Symptoms: Acute Bronchitis
Case no. 6: D. P., Male, Age 21, USA, Tucson Symptoms: Acute Bronchitis
Case no. 7: L. R., Male, Age 26, France, Paris Symptoms: Bronchopneumonia
Case no. 8: G. V., Male, Age 43, USA, Jersey City Symptoms: Pneumonia
Case no. 9: W. Z. Female, Age 21, USA, Long Beach Symptoms: Pneumonia
Case no. 10: G. J. Female, Age 34, USA, San Jose Symptoms: Acute Bronchitis
Case no. 11: W. B. Female, Age 27, Norway, Oslo Symptoms: Bronchopneumonia
Case no. 12: C. R. Female. Age 56, USA, Los Angeles Symptoms: Pneumonia
Case no. 13: O. B. Female, Age 34, USA, Rochester Symptoms: Acute Bronchitis
Case no. 14: Z. F. Female, Age 50, USA, Columbus Symptoms: Bronchopneumonia
Case no. 15: K. U. Female, Age 32, Canada, Toronto Symptoms: Acute Bronchitis

### Additional Information and Results of Testing

The reason for this testing was to check if the novel compound, after a longer period of use, can cause any eventual side effects.

The test results were generated and monitored in order to obtain the relevant information and facts. In this study, the general monitored parameters were: pulse, breathing, ECG (Electrocardiography), discomfort, adverse events, and/or allergy monitoring. These parameters were measured before the treatment with the novel compound and 15 min after its intake. The novel compound was ingested on the therapeutic days in each patient at 06:00 AM, 02:00 PM, and 10:00 PM (Table 1 and Table 2). After the novel compound had been inhaled, the physician made observations for 30 min. The patients' feelings during and after inhaling the novel compound will also be recorded for each participant.

As part of the biochemical analysis of patients' blood, each patient's blood sample was checked for biochemical parameters. Blood samples were taken from each patient before the beginning of the test, on the 4^{th} day from the test beginning, and on the 8^{th} day from the test beginning. The last blood samples have to be checked 12 h from the previous inhalation of the novel compound. The biochemical parameters that were measured in blood samples include:
1. Liver status
2. C Reactive protein
3. Kidney Status

The dose was: 10 mL of the novel compound 2%/30 kg of the body weight every 8 h for seven (7) days (Table 1).

The clinical study started on the 15^{th} of October, 2017 and was completed on the 21^{st} of November, 2017.

Each patient received a dose at 6:00 AM, 2:00 PM, and 10:00 PM each day for seven (7) days.

List of participating male patients and the corresponding doses of the novel compound:

| No. | Patient | Gender | Age (years) | Weight (kg) | Dose (mL) |
|---|---|---|---|---|---|
| 1 | J. P. | M | 27 | 71 | 23.67 |
| 2 | I. N. | M | 31 | 72 | 24.00 |
| 3 | M. S. | M | 23 | 63 | 21.00 |
| 4 | P. S. | M | 29 | 67 | 22.33 |
| 5 | I. J. | M | 37 | 74 | 24.67 |
| 6 | A. V. | M | 20 | 66 | 22.00 |
| 7 | P. S. | M | 24 | 70 | 23.33 |
| 8 | R. M. | M | 32 | 73 | 24.33 |
| 9 | D. N. | M | 39 | 68 | 22.67 |
| 10 | N. S. | M | 37 | 71 | 23.67 |
| 11 | D. S. | M | 28 | 69 | 23.00 |
| 12 | L. V. | M | 40 | 73 | 24.33 |
| 13 | N. A. | M | 22 | 63 | 21.00 |
| 14 | S. M. | M | 25 | 67 | 22.33 |
| 15 | T. S. | M | 21 | 65 | 21.67 |
| 16 | O. L. | M | 33 | 66 | 22.00 |
| 17 | Z. M. | M | 29 | 73 | 24.33 |
| 18 | I. S. | M | 25 | 63 | 21.00 |
| 19 | J. T. | M | 40 | 74 | 24.67 |
| 20 | P. L. | M | 37 | 71 | 23.67 |
| 21 | V. J. | M | 24 | 67 | 22.33 |
| 22 | G. N. | M | 21 | 66 | 22.00 |
| 23 | R. S. | M | 36 | 69 | 23.00 |
| 24 | P. J. | M | 20 | 63 | 21.00 |
| 25 | D. V. | M | 33 | 71 | 23.67 |
| 26 | S. I. | M | 28 | 74 | 24.67 |
| 27 | K. D. | M | 22 | 68 | 22.67 |
| 28 | V. I. | M | 39 | 72 | 24.00 |
| 29 | I. A. | M | 31 | 65 | 21.67 |
| 30 | P. T. | M | 35 | 69 | 23.00 |
| 31 | R. V. | M | 27 | 71 | 23.67 |
| 32 | T. N. | M | 20 | 64 | 21.33 |
| 33 | M. A. | M | 26 | 72 | 24.00 |
| 34 | I. R. | M | 21 | 63 | 21.00 |
| 35 | J. V. | M | 29 | 67 | 22.33 |
| 36 | G. S. | M | 33 | 74 | 24.67 |
| 37 | O. D. | M | 28 | 72 | 24.00 |
| 38 | Z. V. | M | 24 | 68 | 22.67 |
| 39 | V. T. | M | 31 | 73 | 24.33 |
| 40 | S. S. | M | 38 | 74 | 24.67 |

List of participating female patients and the corresponding doses of the novel compound:

| No. | Patient | Gender | Age (years) | Weight (kg) | Dose (mL) |
|---|---|---|---|---|---|
| 1 | M. I. | F | 24 | 58 | 19.33 |
| 2 | J. S. | F | 31 | 63 | 21.00 |
| 3 | M. L. | F | 27 | 61 | 20.33 |
| 4 | A. R. | F | 25 | 65 | 21.67 |
| 5 | I. S. | F | 33 | 57 | 19.00 |
| 6 | L. M. | F | 34 | 64 | 21.33 |
| 7 | P. S. | F | 20 | 62 | 20.67 |
| 8 | T. T. | F | 22 | 59 | 19.67 |
| 9 | R. P. | F | 28 | 64 | 21.33 |
| 10 | I. N. | F | 25 | 61 | 20.33 |
| 11 | J. M. | F | 29 | 67 | 22.33 |
| 12 | Z. N. | F | 23 | 56 | 18.67 |
| 13 | S. V. | F | 34 | 62 | 20.67 |
| 14 | L J. V. | F | 31 | 64 | 21.33 |
| 15 | N. J. | F | 30 | 61 | 20.33 |
| 16 | S. O. | F | 27 | 62 | 20.67 |
| 17 | K. N. | F | 21 | 57 | 19.00 |
| 18 | S. B. | F | 29 | 59 | 19.67 |
| 19 | M. C. | F | 24 | 58 | 19.33 |
| 20 | S. D. | F | 33 | 64 | 21.33 |
| 21 | L. D. | F | 38 | 66 | 22.00 |
| 22 | T. Z. | F | 25 | 62 | 20.67 |
| 23 | P. T. | F | 24 | 67 | 22.33 |
| 24 | L. S. | F | 31 | 61 | 20.33 |
| 25 | E. C. | F | 26 | 58 | 19.33 |
| 26 | A. V. | F | 21 | 61 | 20.33 |
| 27 | D. A. | F | 24 | 68 | 22.67 |
| 28 | R. D. | F | 28 | 71 | 23.67 |
| 29 | I. B. | F | 20 | 62 | 20.67 |
| 30 | O. R. | F | 33 | 64 | 21.33 |
| 31 | K. A. | F | 29 | 68 | 22.67 |
| 32 | I. D. | F | 25 | 61 | 20.33 |
| 33 | P. R. | F | 21 | 57 | 19.00 |
| 34 | E. N. | F | 34 | 66 | 22.00 |
| 35 | U. R. | F | 31 | 68 | 22.67 |
| 36 | A. P. | F | 28 | 65 | 21.67 |
| 37 | Z. A. | F | 23 | 59 | 19.67 |
| 38 | I. G. | F | 21 | 61 | 20.33 |
| 39 | R. B. | F | 29 | 63 | 21.00 |
| 40 | O. K. | F | 31 | 70 | 23.33 |

Based on the results obtained during this clinical pilot study of the compound on 80 humans, the following conclusions can be made:
The study participants showed tolerance to the novel compound upon the prescribed dosage.
There were no allergy symptoms due to the prescribed dosage of the novel compound.
During the inhalation of the novel compound, pulse frequency and blood pressure were not significantly altered.
Electrocardiography measurements during the intake of the novel compound were regular without any pathological and irregular oscillations.
Respiratory function was also monitored during the clinical study. The respiratory function of patients was not disturbed, and none reported any irregularity in respiratory function during the study.
No dyspnea was recorded during the test.
No bronchospasms were recorded.
Sporadic coughing was recorded in some participants at the beginning of the test.
No irregular parameters were reported in the liver function biochemical analysis from blood samples during the study. Therefore, the novel compound had no negative clinical or acute effects on the hepatic function of participants volunteering in this study.
No irregular parameters were reported while testing kidney functions for the period of testing the novel compound. The test results of both urine and blood parameters related to kidney functions have suggested that the novel compound did not have any negative effects on the kidneys of patients.
The results of C Reactive Protein (CRP) measurements during the study with the novel compound suggest no inflammation was developed.
After ten working days from the end of the testing, no irregularities were observed on the tested participants.

### The Compound In Vitro Test

Here are the findings of the comparative examination of selected pathogens against the novel compound and the antibiotics Cephalosporin and Ampicillin:

| Bacteria | Method | Novel Compound | R | Cephalosporin | | R | Ampicilin | R |
|---|---|---|---|---|---|---|---|---|
| Pasteurella | Müler-Hinton-Agar | 36 mm | ES | 12 | mm | S | 5 mm | MS |
| Cryptococcus | Müler-Hinton-Agar | 31 mm | ES | 6 | mm | MS | 0 mm | R |
| Campylobacter | Müler-Hinton-Agar | 32 mm | ES | 11 | mm | S | 7 mm | MS |
| Microsporidia | Müler-Hinton-Agar | 30 mm | ES | 22 | mm | ES | 3 mm | R |
| Histoplasma capsulatum | Müler-Hinton-Agar | 34 mm | ES | 4 | mm | R | 2 mm | R |
| Micrococcus | Müler-Hinton-Agar | 38 mm | ES | 8 | mm | MS | 0 mm | R |
| Candida | Müler-Hinton-Agar | 33 mm | ES | 7 | mm | MS | 4 mm | R |

Amount used: The novel compound 0.05 mL; Cephalosporin 0.1 mL; Ampicillin 0.2 mL.

Evaluation of the results by zone diameter:
0 mm-4 mm: resistant: R
5 mm-9 mm: moderately sensitive: MS
10 mm-15 mm: sensitive: S
16 mm-20 mm: very sensitive: VS
21 mm and more: extremely sensitive: ES

### Testing the Toxicity of the Novel Compound Inhalable Liquid

This testing aimed to determine the acute toxicity—LD50 of the novel compound inhalable liquid in mice and rats. Both mice and rats were specially selected to test the toxicity of the compounds, medicine, and chemicals. Only acute toxicity testing was done since the active therapy with the novel compound takes from one (1) to three (3) days. Sub-acute toxicity was not necessary and applicable since extended treatment with the novel compound will not be in function or planned. Testing was done in the inhalable application in both white lab rats and white lab mice. Groups of 100 lab mice and rats of both genders were placed into glass compartments. They were regularly consuming food manufactured for these lab animals. Water consumption was always available.

Inhalation administration requires more drugs than other routes of administration,

In this toxicology test—the acute toxicity of the novel compound was tested:
Oral LD50 p.o.—mice
Oral LD50 p.o.—rats
Inhalation LD50—mice
Inhalation LD50—rats

### Peroral Application (p.o.) of the Novel Compound—LD50—Testing and Doses

The peroral application (p.o.) acute toxicity LD50 testing was upon the standard procedure: application p.o. with the special lab pipette easy to administer liquid product: orally to the lab animals.

Results of the peroral application (p.o.) testing of LD50 of the novel compound at mice:

Peroral application (p.o.) of the novel compound testing LD50 has been by mice: 1 μL/kg, 5 μL/kg, 10 μL/kg, 20 μL/kg, 50 μL/kg, 100 μL/kg, 150 μL/kg, 200 μL/kg, 300 μL/kg, 400 μL/kg, 500 μL/kg, 600 μL/kg, 700 μL/kg, 800 μL/kg, 900 μL/kg, 1,000 μL/kg, 1,250 μL/kg, 1,500 μL/kg, 1,750 μL/kg, and 2,000 μL/kg. At the level of oral dose LD-50 2,000 μL/kg—twenty-three (23) mice out of a hundred (100) have changed their behavior and demonstrated light discomfort: running as well as anxiety. Breathing and cardiac functions were regular, and the function of the thorax was regular and without visual changes. Discomfort symptoms noticed after the applications disappeared after 1-3 min, diverse from mouse to mouse. No mouse has ended lethally during this test. The conclusion based on these test results was: the novel compound has oral LD50 over 2,000 μL/kg for mice (p.o.). The novel compound is low toxic in the applied doses for mice orally (p.o.).

### Results of the Peroral Application (p.o.) Testing of LD50 of the Novel Compound at Rats:

Peroral application (p.o.) of the novel compound testing LD50 has been by rats: 1 μL/kg, 5 μL/kg, 10 μL/kg, 20 μL/kg, 50 μL/kg, 100 μL/kg, 150 μL/kg, 200 μL/kg, 300 μL/kg, 400 μL/kg, 500 μL/kg, 600 μL/kg, 650 μL/kg, 750 μL/kg, 850 μL/kg, 1,000 μL/kg, 1,250 μL/kg, 1,500 μL/kg, 2,000 μL/kg, and 2,250 μL/kg. At the level of oral dose LD50 2,250 μL/kg—thirty-seven (37) rats out of hundred (100) have changed their behavior and demonstrated light discomfort: running over the glass cubical, as well as anxiety. Breathing and cardiac functions were regular, function of the thorax was regular and without visual changes. Discomfort symptoms noticed after the applications disappeared after 1-3 min, diverse from rat to rat. No rats ended lethally during this test. The conclusion based on these test results was: the novel compound has oral LD50 over 2,250 μL/kg for rats (p.o.). The novel compound is low toxic in the applied doses for rats orally (p.o.).

### Inhalatory Application of the Novel Compound—LD50—Testing and Doses

Inhalatory testing of the novel compound LD50 by nose-only exposure, designed for the easy application of the novel compound to the lab animals through inhalation. The inhalation process was conducted using a nebulizer with tubes going directly into the nostrils of the animals. The exposure time for laboratory rats was 6 h for each test and 4 h for laboratory mice.

Results of inhalatory application of the novel compound in mice: Inhalatory application the novel compound testing LD50 has been in mice: 1 μL/kg, 5 μL/kg, 10 μL/kg, 20 μL/kg, 50 μL/kg, 100 μL/kg, 150 μL/kg, 200 μL/kg, 300 μL/kg, 400 μL/kg, 500 μL/kg, 600 μL/kg, 700 μL/kg, 800 μL/kg, 900 μL/kg, 1,000 μL/kg, 1,250 μL/kg, 1,500 μL/kg, 1,750 μL/kg, 2,000 μL/kg, and 2,250 μL/kg. At the level of i.p. dose LD50 2,250 μL/kg—twenty-one (21) mice out of hundred (100) have changed their behavior and demonstrated discomfort: running as well as anxiety. Breathing and cardiac functions were regular, function of the thorax was regular and without visual changes. Discomfort symptoms noticed after the applications disappeared after 3-5 min, diverse from mouse to mouse. No mouse has ended lethally during this test. The conclusion based on these test results was: the novel compound inhalable liquid has LD50 over 2,250 μL/kg for mice. The novel compound is low toxic in the applied doses on mice. The animals were reexamined after 14 days, and no new irregularities were found.

Results of inhalatory application of the novel compound in rats: Inhalatory application of the novel compound testing LD50 has been by rats: 1 μL/kg, 5 μL/kg, 10 μL/kg, 20 μL/kg, 50 μL/kg, 100 μL/kg, 150 μL/kg, 200 μL/kg, 300 μL/kg, 4100 μL/kg, 500 μL/kg, 600 μL/kg, 700 μL/kg, 800 μL/kg, 900 μL/kg, 1,000 μL/kg, 1,250 μL/kg, 1,500 μL/kg, 1,750 μL/kg, 2,000 μL/kg, 2,250 μL/kg, and 2,500 μL/kg. At the level of i.p. dose LD50 2.500 μL/kg—twenty-nine (29) rats out of hundred (100), were changed their behavior and demonstrated discomfort: running over the glass cubical as well as the anxiety. Breathing and cardiac functions were regular, function of the thorax was regular and without visual changes. Discomfort symptoms were noticed after the applications disappeared after 5-6 min, diverse from rat to rat. No rat has ended lethally during this test. The conclusion based on these test results was: the novel compound inhalable liquid has LD50 over 2,500 μL/kg for rats. The novel compound is low toxic in the applied doses on rats. The animals were reexamined after 14 days, and no new irregularities were found

### Mutagenic and Cancer-Genic Tests of the Novel Compound Inhalable Liquid

The subject of the testing was to determine the mutagenic and carcinogenic effect of the novel compound inhalable liquid has been conducted. The test has been done using the Ames test. This test, which is a widely and pretty often used method that uses pathogens, in most cases bacteria, to test whether a given chemical, compound, and/or medicine can damage the tested organism's DNA. This method is fast to conduct and easy to follow to check the tested compound's mutagenic or carcinogenic effect. The lab dish with a specially selected Candida strain requiring histidine was added to a rat liver extract. The test has been done using the control experimental method. In the dish volume of the 20 mL of liquid considered a control, one did not have any of the novel compound liquid. In the dish we consider to be the experimental one, 1 mL of the novel compound was added to the 20 mL of liquid in the dish. The mutagenic test results were negative in both lab dishes—control and experimental plates. The results were identical, with no signs of the mutations. Based on these preliminary results, a conclusion could be made that the novel compound possesses no mutagenic and/or cancerogenic effects.

What is claimed:

1. A compound manufactured with the procedure of synthesis of bouncing Eugenol and Lidocaine into a stabile compound having a structure as shown in Formula I:

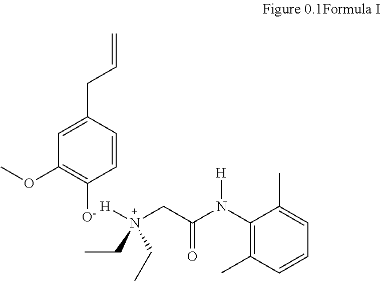

2. Use of the compound, in the form of an inhaled liquid according to the patent claim 1, as an active compound/potent antimicrobial for the treatment of respiratory infections in humans caused by pathogens resistant to antibiotics, such as Pasteurella spp., Cryptococcus spp., Campylobacter spp., Microsporidia spp., Histoplasma capsulatum, Micrococcus spp., Candida spp., and other bacteria.
